(19) United States
(12) Patent Application Publication
(10) Pub. No.: US 2015/0337031 A1
(43) Pub. Date: Nov. 26, 2015

(54) BIOMARKER METHODS AND COMPOSITIONS

(71) Applicant: CELYAD S.A., Mont-Saint-Guibert (BE)

(72) Inventors: Sebastien MAUEN, Mont-Saint-Guibert (BE); Peter DE WAELE, Mont-Saint-Guibert (BE); Roldand GORDON-BERESFORD, La Hulpe (BE)

(21) Appl. No.: 14/654,211

(22) PCT Filed: Dec. 19, 2013
(86) PCT No.: PCT/EP2013/077476

§ 371 Date: Jun. 19, 2015

(30) Foreign Application Priority Data
Dec. 20, 2012 (GB) 1223058.7

(51) Int. Cl. C07K 16/18; G01N 33/68

(52) U.S. Cl. CPC C07K 16/18; G01N 33/6878; C07K 2317/34; C07K 2317/92

(57) ABSTRACT

Antibodies are provided that bind selectively to MEF2C (Myocyte-specific enhancer factor 2C)—also known as MADS box transcription enhancer factor 2, polypeptide C. MEF2C is one of several biomarkers expressed in committed cells differentiated from stem cells. MEF2C is a transcription factor in the MEF2 family of proteins, which play a role in cardiac morphogenesis, myogenesis and also vascular development. Also provided are hybridoma that produces the antibodies, as well as methods of use and kits for using the antibodies for diagnostic and therapeutic purposes.

### FIELD

The present invention relates to methods and compositions for identifying a biomarker, in particular for identifying a biomarker of differentiation, especially following the cardiovascular or neural lineages and committed cells obtained by the differentiation of stem cells.

### BACKGROUND

Cardiovascular diseases are a leading cause of mortality worldwide and in contrast to tissues with high reparative capacity, heart tissue is vulnerable to irreparable damage. As such, cell-based regenerative cardiovascular medicine is being pursued to address heart disease disorders. Adult stem cells, delivered in their naïve state, have demonstrated a limited benefit in patients with heart disease disorders so therapies are being developed to guide naïve human stem cells towards a cardiac lineage prior to injection into a patient to aid heart regeneration.

Cardiovascular lineage committed cells express several different biomarkers that can be used to identify when a naïve stem cell has differentiated to a cardiac lineage committed state. One such biomarker is MEF2C (Myocyte-specific enhancer factor 2C), a transcription factor of the MEF2 family that plays an important role in cardiac morphogenesis, myogenesis and vascular development. MEF2C is also known as an important factor in neurogenesis and brain development.

Antibodies for the detection of MEF2C are commercially available but suffer from disadvantages. For example, rabbit polyclonal antibodies obtained through immunisation of rabbits with full length protein or a major protein portion of MEF2C are known. However, these polyclonal antibodies suffer from poor selectivity because the use of the full length protein or a protein portion for immunisation means that the antibodies may be selective for other members of the MEF2 family in addition to MEF2C. Furthermore, polyclonal antibodies suffer from batch-to-batch variations, which means there is no guaranteed consistent level of selectivity or specificity, making these antibodies unsuitable for accurately detecting biomarkers.

Mouse monoclonal antibodies generated through the immunisation of mice with full length protein or a protein portion of MEF2C are also commercially available.

However, these monoclonal antibodies also suffer from poor selectivity because the use of full length protein or a protein portion for immunisation means that the antibodies may be selective for other members of the MEF2 family in addition to MEF2C.

Thus, there is a significant need for highly sensitive and specific detection methods and compositions, in particular for highly specific and sensitive antibodies, that will allow for the accurate detection of lineage committed cells that express MEF2C.

### SUMMARY

In a first aspect, the invention resides in an isolated antibody, or a fragment thereof, that binds selectively to a MEF2C epitope that comprises an amino acid sequence selected from at least one of: SEQ ID NO: 3 and SEQ ID NO: 4. The isolated antibody, or fragment thereof, is suitably human MEF2C. According to an embodiment of the invention the antibody, or fragment thereof, comprises the amino acid sequence of SEQ ID NO: 2. According to an embodiment of the invention the antibody is a monoclonal antibody. Optionally the antibody is an anti-anti-idiotypic antibody.

In accordance with a further embodiment of the invention the monoclonal antibody may be selected from the group consisting of: a mouse monoclonal antibody; a rabbit monoclonal antibody; and a rat monoclonal antibody. It is an option that the isolated antibody, or fragment thereof, is a single domain antibody.

In an alternative embodiment of the invention, the fragment is may be selected from: an scFv fragment, an scFv₂ fragment, an Fv fragment, an Fab fragment, an Fab′ fragment, or an F(ab′)₂ fragment.

A second aspect of the invention provides a hybridoma cell line as deposited with the Belgian Coordinated Collections of Micro-organisms (BCCM), under accession number LMBP 9949CB that produces the antibody as described herein. According to one embodiment of the invention, there is provided a monoclonal antibody, or fragment thereof, produced by the aforementioned deposited hybridoma cell line.

A third aspect of the invention provides a monoclonal antibody, or fragment thereof, that is able to compete with a monoclonal antibody, or fragment thereof, produced by the hybridoma cell line as deposited with the BCCM, under accession number LMBP 9949CB, for specific binding to a MEF2C epitope. Suitably the epitope consists of an amino acid sequence selected from at least one of: SEQ ID NO: 3 and SEQ ID NO: 4. In an embodiment of the invention the monoclonal antibody, comprises a heavy chain that comprises a complementarity-determining region (CDR) selected from at least one or more of SEQ ID NOs: 13-15, and a light chain that comprises a CDR selected from at least one or more of SEQ ID NOs: 16-18.

A fourth aspect of the invention provides a nucleic acid sequence encoding the isolated antibody, or fragment thereof, as described herein. Suitably the nucleic acid sequence comprises a cDNA sequence.

A fifth aspect of the invention provides a nucleic acid sequence that encodes at least one complementarity determining region (CDR) of the isolated antibody, or fragment thereof, as described herein.

A sixth aspect of the invention provides a nucleic acid as described herein, operably linked to a promoter. A seventh aspect provides for an isolated expression vector comprising any of the aforementioned nucleic acids of the invention. An eighth aspect of the invention provides an isolated host cell transformed with the expression vector described herein.

A ninth aspect of the invention provides use of a MEF2C epitope comprising (or consisting essentially of) an amino acid sequence selected from at least one of: SEQ ID NO: 3 and SEQ ID NO: 4 in the production of an antibody. A tenth aspect of the invention provides for the use of a MEF2C epitope comprising (or consisting essentially of) an amino acid sequence selected from at least one of: SEQ ID NO: 3 and SEQ ID NO: 4 for the purpose of screening a peptide display library. In a specific embodiment of the invention the peptide display library is a human combinatorial antibody library (HuCAL®).

An eleventh aspect of the invention provides a composition comprising the antibody, or fragment thereof, according to any aspect as described herein and a carrier. The carrier may suitably comprise a solvent, buffer or preservative solution.

A twelfth aspect of the invention provides a method for detecting a cardiovascular lineage committed cell, comprising:

contacting a biological sample that comprises cells with an antibody, or fragment thereof, that binds selectively to a MEF2C epitope that comprises an amino acid sequence selected from at least one of: SEQ ID NO: 3 and SEQ ID NO: 4, under conditions where an immune complex will form between the antibody, or fragment thereof, and a target antigen; and

detecting the presence of the immune complex;

wherein the presence of the immune complex indicates the presence of a cardiovascular lineage committed cell.

In a specific embodiment of the invention any and all of the disclosed methods may further comprise the step of visualising localisation of the immune complex within the cell. Optionally, the visualisation step is carried out using a technique selected from the group consisting of: light microscopy; UV microscopy; and confocal microscopy.

A thirteenth aspect of the invention provides a method for quantifying a cardiovascular lineage commitment of cells, comprising:

contacting a biological sample that comprises cells with an antibody, or fragment thereof, that binds selectively to a MEF2C epitope that comprises an amino acid sequence selected from at least one of: SEQ ID NO: 3 and SEQ ID NO: 4, under conditions where an immune complex will form between the antibody, or fragment thereof, and a target antigen; and quantifying the presence of the immune complex;

wherein the presence of the immune complex indicates the quantification of cardiovascular lineage commitment of a cell.

Suitably the biological sample may be obtained from a human.

In a specific embodiment of the invention, the method further comprises:

contacting the biological sample with a second antibody that specifically binds the antibody, or fragment thereof. Optionally, the second antibody is coupled to a detectable agent. Typically, the detectable agent comprises one or more of the group selected from: an enzyme; a fluorescent label; a luminescent label; a radioactive label; or a chromogenic label.

A fourteenth aspect of the invention provides a method for detecting a neural lineage committed cell, comprising:

contacting a biological sample that comprises cells with an antibody, or fragment thereof, that binds selectively to a MEF2C epitope that comprises an amino acid sequence selected from at least one of: SEQ ID NO: 3 and SEQ ID NO: 4, under conditions where an immune complex will form between the antibody, or fragment thereof, and a target antigen; and

detecting the presence of the immune complex;

wherein the presence of the immune complex indicates the presence of a neural lineage committed cell.

A fifteenth aspect of the invention provides a method for quantifying a neural lineage commitment of cells, comprising:

contacting a biological sample that comprises cells with an antibody, or fragment thereof, that binds selectively to a MEF2C epitope that comprises an amino acid sequence selected from at least one of: SEQ ID NO: 3 and SEQ ID NO: 4, under conditions where an immune complex will form between the antibody, or fragment thereof, and a target antigen; and

quantifying the presence of the immune complex;

wherein the presence of the immune complex indicates the quantity of a neural lineage commitment of a cell.

In an embodiment of the invention the method is for detecting a neuronal lineage committed cell.

Sixteenth and seventeenth aspects of the invention provides a kit for the identification of a cardiovascular or neural lineage committed cell, respectively, the kit comprising at least one of:

an antibody, or a fragment thereof, that binds selectively to a MEF2C epitope that comprises an amino acid sequence selected from at least one of: SEQ ID NO: 3 and SEQ ID NO: 4.; or

a monoclonal antibody, or fragment thereof, that is able to compete with a monoclonal antibody, or fragment thereof, produced by the hybridoma cell line as deposited with the BCCM, under accession number LMBP 9949CB, for specific binding to a MEF2C epitope, which epitope consists of an amino acid sequence selected from at least one of: SEQ ID NO: 3 and SEQ ID NO: 4; and

instructions for using the kit.

An eighteenth aspect of the invention provides a method of treating an individual having a heart disorder, comprising;

obtaining cells and/or stem cells from an individual;

differentiating the cells and/or stem cells towards a cardiovascular lineage;

detecting whether the cells and/or stem cells have become cardiovascular lineage committed cells using an antibody, or a fragment thereof, that binds selectively to a MEF2C epitope that comprises an amino acid sequence selected from at least one of: SEQ ID NO: 3 and SEQ ID NO: 4;

isolating the cardiovascular lineage committed cells; and

administering the cardiovascular lineage committed cells to an individual in need thereof.

A nineteenth aspect of the invention provides a method of treating an individual having a neurodegenerative disorder, comprising;

obtaining cells and/or stem cells from an individual;

differentiating the cells and/or stem cells towards a neural lineage;

detecting whether the cells and/or stem cells have become neural lineage committed cells using an antibody, or a fragment thereof, that binds selectively to a MEF2C epitope that comprises an amino acid sequence selected from at least one of: SEQ ID NO: 3 and SEQ ID NO: 4;

isolating the neural lineage committed cells; and

administering the neural lineage committed cells to an individual in need thereof.

A twentieth aspect of the invention provides an antibody, or a fragment thereof, that binds selectively to a MEF2C epitope that comprises an amino acid sequence selected from at least one of: SEQ ID NO: 3 and SEQ ID NO: 4, for use in a method of treating heart disease.

A twenty-first aspect of the invention provides a monoclonal antibody, or fragment thereof, that is able to compete with a monoclonal antibody, or fragment thereof, produced by the hybridoma cell line as deposited with the BCCM, under accession number LMBP 9949CB, for specific binding to a MEF2C epitope, which epitope consists of an amino acid sequence selected from at least one of: SEQ ID NO: 3 and SEQ ID NO: 4, for use in a method of treating heart disease. Suitably the monoclonal antibody may comprise a heavy chain that comprises a complementarity-determining region (CDR) selected from at least one or more of SEQ ID NOs: 13-15, and the light chain may comprise a CDR selected from at least one or more of SEQ ID NOs: 16-18.

These methods may comprise use of the antibody to detect and facilitate isolation of cells that are committed to a cardiovascular lineage, for use in cellular therapy.

It will be appreciated that where appropriate any of the above embodiments and aspects of the invention may also be used in combination.

### BRIEF DESCRIPTION OF THE DRAWINGS

FIG. 1 shows the results of immunodetection using Western blotting on empty vector (−) and MEF2C coding vector (+) transfected cells. The anti-MEF2C monoclonal antibody (α MEF2C) of the present invention recognises a specific band in the MEF2C transfected cells (+) at around 60 kDa—this corresponds to MEF2C.

FIG. 2, left panel, shows the results of MEF2C immunostaining in transfected HeLa cells. The right panel shows the distribution of MEF2C and Flag staining in untransfected (+) and MEF2C transfected (x) HeLa cells. Yellow crosses (x) in the top right quadrant of the graph represent double positive nuclei, i.e. cells that show staining intensities for both MEF2C and Flag.

FIG. 3 shows the immunofluorescence of the bone marrow mesenchymal stem cells (left picture) compared to cardiovascular lineage committed cells from the same bone marrow origin (right picture).

FIG. 4 is a mean nuclear intensity representation of bone marrow mesenchymal stem cell nuclei and cardiovascular lineage committed cell nuclei.

FIG. 5 is a histogram of nuclear fluorescence intensity versus cell population proportion with fluorescence intensity intervals of 7.5 units. Bone marrow mesenchymal cells are represented by the grey front row of bars. Cardiac lineage committed cells are represented by the red back row of bars.

FIG. 6 shows the consensus sequence of the anti-MEF2C antibody produced by a hybridoma according to the present invention.

FIG. 7 shows a histogram of measured absorbance at 450 nm showing the relative importance of residues within the MEF2C epitope for anti-MEF2C binding in an alanine scanning epitope mapping study. Different single point mutations of the wild-type peptide and either anti-MEF2C mAb (dark grey bars) or anti-GST mAb (light grey bars). Wild type peptide was used as positive control for anti-MEF2C mAb mediated detection and GST-peptide for anti-GST mAb mediated detection.

FIG. 8 shows a histogram in which relative quantification of binding of anti-MEF2C monoclonal antibody in an alanine scanning epitope mapping study is measured.

### DETAILED DESCRIPTION

Unless otherwise indicated, the practice of the present invention employs conventional techniques of chemistry, molecular biology, microbiology, recombinant DNA technology, chemical methods, pharmaceutical formulations and delivery and treatment of patients, which are within the capabilities of a person of ordinary skill in the art. Such techniques are also explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Prior to setting forth the invention, a number of definitions are provided that will assist in the understanding of the invention.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “amino acid” as used herein includes naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The term “amplification” refers to use of a technique that increases the number of copies of a nucleic acid molecule in a sample, for example the amplification of a nucleic acid that encodes the antibody of the present invention, or fragment thereof. An example of amplification is the polymerase chain reaction, in which a biological sample collected from a subject is contacted with a pair of oligonucleotide primers, under conditions that allow for the hybridisation of the primers to a nucleic acid template in the sample. The primers are extended under suitable conditions, dissociated from the template, and then re-annealed, extended, and dissociated to amplify the number of copies of the nucleic acid. The product of amplification may be characterised by electrophoresis, restriction endonuclease cleavage patterns, oligonucleotide hybridisation or ligation, and/or nucleic acid sequencing using standard techniques.

The term “antibody” denotes a polypeptide ligand comprising at least a light chain or heavy chain immunoglobulin variable region which specifically recognises and binds an epitope of an antigen or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognised by the antibody. This includes intact immunoglobulins and the variants and portions of them well known in the art, such as Fab fragments, Fab′ fragments, F(ab)′₂ fragments, Fv fragments, single chain Fv proteins (“scFv”), scFv₂ fragments and disulfide stabilised Fv proteins (“dsFv”). A scFv protein is a fusion protein in which a light chain variable region of an immunoglobulin and a heavy chain variable region of an immunoglobulin are bound by a linker, while in dsFv, the chains have been mutated to introduce a disulfide bond to stabilise the association of the chains. The term also includes genetically engineered forms such as chimeric antibodies (for example, humanised murine antibodies), heteroconjugate antibodies (such as, bispecific antibodies), anti-anti-idiotypic antibodies as well as single domain antibodies including nanobodies or engineered, genetic as well as in silico, constructs based on the primary, secondary, tertiary or quaternary structure. See also, Pierce Catalogue and Handbook, 1994-1995 (Pierce Chemical Co., Rockford, Ill.); Kuby, J., Immunology, 3^{rd} Ed., W.H. Freeman & Co., New York, 1997. Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The sequences of the framework regions of different light or heavy chains are relatively conserved within a species. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. An antibody that binds an antigen of interest has a specific V_{H} region and V_{L} region sequence, and thus specific CDR sequences. Antibodies with different specificities (due to different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). The term “antibody” also includes any portion of an antibody having specificity towards at least one desired epitope that competes with the intact antibody for specific binding (e.g. a fragment having sufficient CDR sequences and having sufficient framework sequences so as to bind specifically to an epitope). By way of example, an antigen binding fragment can compete for binding to an epitope which binds the antibody from which the fragment was derived.

The specificity of an antibody for a given epitope can be determined by way of a competition assay (see, for example, Stahli C, Miggiano V, Stocker J, Staehelin T, Haring P, Takács B (1983) Methods Enzymol; 92:242-53).

The term “monoclonal antibody” denotes an antibody produced by a single clone of hybridoma cells or by a cell into which the light and heavy chain genes of a single antibody have been transfected, or a progeny thereof. Monoclonal antibodies are produced by methods known to those of skill in the art, for instance by making hybrid antibody-forming cells (hybridoma cells) from a fusion of myeloma cells with immune spleen cells.

The term “chimeric antibody” denotes an antibody having framework residues from one species, such as human, and CDRs or SDRs (which generally confer antigen binding) from another species. Most typically, chimeric antibodies include human and murine antibody domains, generally human constant regions and/or framework regions and murine variable regions, murine CDRs and/or murine SDRs.

The term “humanised antibody” denotes an immunoglobulin including a human framework region and one or more CDRs from a non-human or SDRs (for example a mouse, rat, or synthetic) immunoglobulin. The non-human immunoglobulin providing the CDRs is termed a “donor,” and the human immunoglobulin providing the framework is termed an “acceptor.”

Human antibodies can be made by introducing human immunoglobulin loci into transgenic animals, such as mice, in which the endogenous immunoglobulin genes have been partially or completely inactivated. Upon immunological challenge, human antibody production is observed in the host, which closely resembles that seen in humans in all respects, including gene rearrangement, assembly, and antibody repertoire.

“Single domain antibodies” refer to an antibody fragment that comprises a single monomeric antigen binding (i.e. variable) domain. Single domain antibodies, sometimes referred to as Nanobodies, may comprise single domain heavy chain antibodies of non-human origin such as camelid V_{H}H antibody fragments. Such antibody fragments are typically smaller in size than conventional antibodies or even Fab fragments.

The term “antigen” denotes a molecule that triggers an immune response. An antigen may be in the form of a full length polypeptide or protein. Alternatively, the antigen can be in the form of peptide fragments that bear the specific epitopes that allow antibodies raised against such fragments to also bind to the full length polypeptide.

The term “differentiation” as used herein denotes the process by which a less-specialised cell becomes a more specialised cell.

The term “expression vector” is used to denote a DNA molecule that is either linear or circular, into which another DNA sequence fragment of appropriate size can be integrated. Such DNA fragment(s) can include additional segments that provide for transcription of a gene encoded by the DNA sequence fragment. The additional segments can include and are not limited to: promoters, transcription terminators, enhancers, internal ribosome entry sites, untranslated regions, polyadenylation signals, selectable markers, origins of replication and such like. Expression vectors are often derived from plasmids, cosmids, viral vectors and yeast artificial chromosomes; vectors are often recombinant molecules containing DNA sequences from several sources.

The term “host cell” denotes a cell in which a vector can be propagated and its DNA expressed, for example a vector encoding a disclosed antibody of fragment thereof. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. A host cell may propagate a vector encoding the antibody of the present invention, a functional fragment thereof, or a humanised form thereof.

The term “isolated”, when applied to a polynucleotide sequence, denotes that the sequence has been removed from its natural organism of origin and is, thus, free of extraneous or unwanted coding or regulatory sequences. The isolated sequence is suitable for use in recombinant DNA processes and within genetically engineered protein synthesis systems. Such isolated sequences include cDNAs and genomic clones. The isolated sequences may be limited to a protein encoding sequence only, or can also include 5′ and 3′ regulatory sequences such as promoters and transcriptional terminators.

The term “isolated”, when applied to a polypeptide is a polypeptide that has been removed from its natural organism of origin. It is preferred that the isolated polypeptide is substantially free of other polypeptides native to the proteome of the originating organism. It is most preferred that the isolated polypeptide be in a form that is at least 95% pure, more preferably greater than 99% pure. In the present context, the term “isolated” is intended to include the same polypeptide in alternative physical forms whether it is in the native form, denatured form, dimeric/multimeric, glycosylated, crystallised, or in derivatised forms.

The term “label” denotes a detectable compound or composition that is conjugated directly or indirectly to another molecule, such as an antibody or a protein, to facilitate detection of that molecule. Specific, non-limiting examples of labels include fluorescent tags, enzymatic linkages, luminescent tags and radioactive isotopes.

The term “operably linked”, when applied to DNA sequences, for example in an expression vector, indicates that the sequences are arranged so that they function cooperatively in order to achieve their intended purposes, i.e. a promoter sequence allows for initiation of transcription that proceeds through a linked coding sequence as far as the termination sequence.

A “polynucleotide” is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Polynucleotides include DNA and RNA, and may be manufactured synthetically in vitro or isolated from natural sources. Sizes of polynucleotides are typically expressed as the number of base pairs (bp) for double stranded polynucleotides, or in the case of single stranded polynucleotides as the number of nucleotides (nt). One thousand by or nt equal a kilobase (kb). Polynucleotides of less than around 40 nucleotides in length are typically called “oligonucleotides”.

A “polypeptide” is a polymer of amino acid residues joined by peptide bonds, whether produced naturally or in vitro by synthetic means. Polypeptides of less than around 12 amino acid residues in length are typically referred to as a “peptides”. The term “polypeptide” as used herein denotes the product of a naturally occurring polypeptide, precursor form or proprotein. Polypeptides also undergo maturation or post-translational modification processes that may include, but are not limited to: glycosylation, proteolytic cleavage, lipidisation, signal peptide cleavage, propeptide cleavage, phosphorylation, and such like. A “protein” is a macromolecule comprising one or more polypeptide chains.

The term “promoter” as used herein denotes a site on DNA to which RNA polymerase will bind and initiate transcription. Promoters are commonly, but not always, located in the 5′ non-coding regions of genes.

The term “specific binding” as used herein denotes the selective interaction of an antibody or fragment thereof with an antigen or epitope. For such an interaction to be selective, the antibody will not substantially bind, or can be made to not substantially bind, to markers other than the particular antigen.

The term “cell” as used herein denotes any cell of the human or animal body which can be used to be differentiated, redifferentiated as well as dedifferentiated by any means, including e.g. iPS cells (induced Pluripotent Stem cells).

The term “stem cell” as used herein denotes an unspecialised cell characterised by the ability to self-renew by mitosis while in an undifferentiated state and having the capacity to give rise to various differentiated cell types by cell differentiation. In mammals, there are two broad types of stem cells: embryonic stem cells, which are isolated from the inner cell mass of blastocysts; and adult stem cells, which are found in various tissues. Adult stem cells act as a repair system replacing tissues damaged by disease or injury. They also maintain the normal turnover of regenerative organs such as skin, blood and intestinal tissues. Embryonic stem cells may differentiate into all of the specialised embryonic tissues.

Differentiation of Stem Cells into Cardiovascular Lineage Committed Cells

Adult stem cells, delivered in their naïve state, have demonstrated a limited benefit in patients with heart disease disorders so therapies are being developed to guide naïve human stem cells towards a cardiac lineage prior to injection into a patient to aid heart regeneration. Of increased interest are adult mesenchymal stem cells due to their accessibility for harvest, their propensity to propagate in culture and their favourable biological profile.

Stem cells are biological cells found in all multicellular organisms that have the ability to divide (through mitosis) and differentiate into diverse specialised cell types and can self-renew to produce more stem cells. Cardiogenesis is the process by which stem cells develop into cardiac cells to form the heart muscle. cardiovascular precursor cells, also known as cardiopoietic cells or cardiovascular progenitor cells, are cells which are committed to the cardiac lineage but are not yet fully differentiated into cardiomyocytes or cardiac endothelial cells or smooth muscle cells. It is desirable to be able to detect and isolate cardiopoietic cells, since these cells have greater potential for use in regenerative therapy than e.g. fully differentiated cardiomyocytes.

Cardiac development is controlled by an evolutionarily conserved network of transcription factors, including those encoded by the genes Nkx2.5, GATA4, GATA6, MEF2C, Tbx5, Mesp1, FOG1, FOG2 and Flk1, which connect signalling pathways with genes for muscle growth, contractility and patterning.

Cardiopoietic cells have a specific phenotype and are characterised by the nuclear translocation of the early cardiac transcription factor Nkx2.5 and the late cardiac transcription factor MEF2C, combined with an absence of detectable sarcomeric proteins. Non-detectable levels of sarcomeric protein expression is a specific feature of cardiopoietic cells, which distinguishes them from other cardiomyocyte-like cells derived from stem cells. The nuclear translocation of Nkx2.5 and MEF2C polypeptides is necessary for definitive cardiac lineage commitment.

Cardiopoietic cells can be derived from stem cells including, for example, adult stem cells, embryonic stem cells, induced pluripotent stem cells (IPS), marrow-isolated adult multilineage inducible cells (MIAMI), resident cardiac stem cells, or any combination thereof. Suitably, the stem cells are mesenchymal stem cells harvested from any suitable tissue source including, for example, bone marrow, adipose tissue, umbilical cord blood, amniotic fluid, menstrual fluid, blood. Suitably, cells committed to the generation of heart tissue are mammalian cells, typically selected from the group consisting of humans, cats, dogs, pigs, horses, mice, rats, hamsters and other mammals.

Stem cells may be guided towards a cardiac lineage by contacting the cells with a cocktail of growth factors, cytokines, hormones and combinations thereof. Such substances may be selected in the group consisting of: bone morphogenetic proteins (BMP) such as BMP-1, BMP-2, BMP-5, BMP-6; epidermal growth factor (EGF); erythropoietin (EPO); fibroblast growth factors (FGF) such as FGF-1, FGF-4, FGF-5, FGF-12, FGF-13, FGF-15, FGF-20; granulocyte-colony stimulating factor (G-CSF); granulocyte-macrophage colony stimulating factor (GM-CSF); growth differentiation factor-9 (GDF-9); hepatocyte growth factor (HGF); insulin-like growth factor (IGF) such as IGF-2; myostatin (GDF-8); neurotrophins such as NT-3, NT-4, NT-1 and nerve growth factor (NGF); platelet-derived growth factor (PDGF) such as PDGF-beta, PDGF-AA, PDGF-BB; thrombopoietin (TPO); transforming growth factor alpha (TGF-α); transforming growth factors β (TGF-β) such as TGF-β1, TGF-β2, TGF-β3; vascular endothelial growth factor (VEGF) such as VEGF-A, VEGF-C; TNF-α; leukaemia inhibitory factor (LIF); interleukin 6 (IL-6); retinoic acid; stromal cell-derived factor-1 (SCDF-1); brain-derived neurotrophic factor (BDNF); periostin; angiotensin II; Flt3 ligand; glial-derived neurotrophic factor; heparin; insulin-like growth factor binding protein-3; insulin-like growth factor binding protein-5; interleukin-3; interleukin-8; midkine; progesterone; putrescine; stem cell factor; Wnt1; Wnt3a; Wntδa; caspase-4; chemokine ligand 1; chemokine ligand 2; chemokine ligand 5; chemokine ligand 7; chemokine ligand 11; chemokine ligand 20; haptoglobin; lectin; cholesterol 25-hydroxylase; syntaxin-8; syntaxin-11; ceruloplasmin; complement component 1; complement component 3; integrin alpha 6; lysosomal acid lipase 1; 13-2 microglobulin; ubiquitin; macrophage migration inhibitory factor; cofilin; cyclophillin A; FKBP12; NDPK; profilin 1; cystatin C; calcyclin; stanniocalcin-1; PGE-2; mpCCL2; IDO; iNOS; HLA-G5; M-CSF; angiopoietin; PIGF; MCP-1; extracellular matrix molecules; CCL2 (MCP-1); CCL3 (MIP-1α); CCL4 (MIP-1β); CCL5 (RANTES); CCL7 (MCP-3); CCL20 (MIP-3α); CCL26 (eotaxin-3); CX3CL1 (fractalkine); CXCL5 (ENA-78); CXCL11 (i-TAC); CXCL1 (GROα); CXCL2 (GROβ); CXCL8 (IL-8); CCL10 (IP-10); and combinations thereof.

A variety of different cardiogenic cocktails may be used. For example, a cocktail of cardiogenic substances that comprises TGFβ-1, BMP4, α-thrombin, a compound selected from the group consisting of Cardiotrophin and IL-6, and a compound selected from the group consisting of Cardiogenol C and retinoic acid may be used. Another cocktail may comprise TGFβ-1, BMP4, α-thrombin, Cardiotrophin and Cardiogenol C. Yet another cocktail may comprise at least one compound selected from the group consisting of FGF-2, IGF-1, Activin-A, TNF-α, FGF-4, LIF, VEGF-A and combinations thereof. Cocktails may also comprise FGF-2, IGF-1 and Activin-A. Other preferred cocktails comprise Activin-A, FGF-2, IL-6, IGF-1 and retinoic acid. Other cocktails can lack at least one compound chosen in the group consisting of TNF-α, FGF-4, LIF, and VEGF-A.

Particularly preferred cardiogenic cocktails and compositions of these cocktails are disclosed in International Patent Publication Nos. WO 2010/133686 and WO 2009/151907, which are incorporated by reference, and include TGFβ-1, BMP4, FGF2, IGF-1, Activin-A, Cardiotrophin, α-thrombin and Cardiogenol C in order to derive a cardiopoietic population of cells. Bone marrow mesenchymal stem cells contacted with these cardiogenic cocktails demonstrate differentiation towards a cardiac lineage and may be transplanted into humans to aid in heart tissue regeneration. Such cocktails may be used in the present invention to derive cardiovascular lineage committed cells from human bone marrow mesenchymal stem cells, although any appropriate method can be used to derive cardiovascular lineage committed cells from stem cells for use with the compositions and methods of the present invention.

MEF2C

MEF2C (Myocyte-specific enhancer factor 2C)—also known as MADS box transcription enhancer factor 2, polypeptide C—is one of several biomarkers expressed in committed cells differentiated from stem cells.

MEF2C,a transcription factor of the MEF2 family of proteins, plays a role in cardiac morphogenesis, myogenesis and also vascular development. Transcription factors are proteins that bind to specific DNA sequences and control the transcription of DNA to mRNA. Transcription factors perform this function alone or with other proteins in a complex and are able to promote or block the recruitment of RNA polymerase, which is the enzyme that performs the transcription of DNA to RNA, to specific genes. Vertebrates have four versions of the MEF2 gene and human versions are denoted as MEF2A, MEF2B, MEF2C and MEF2D. All are expressed in distinct but overlapping patterns during embryogenesis and through adulthood.

The mammalian MEF2 genes share approximately 50% overall amino acid identity and about 95% similarity throughout the highly conserved N-terminal MADS-box domain and Mef2 DNA-binding domain, but their sequences diverge in the C-terminal trans-activation domain.

The mature MEF2C protein is found in the cell nucleus and its level of expression is highest during the early stages of post-natal development. MEF2C forms a complex with class II histone deacetylases in undifferentiated cells but upon myogenic differentiation, the histone deacetylases are released into the cytoplasm allowing MEF2C to interact with other proteins for activation.

The encoded human MEF2C protein is 473 amino acids in length and three isoforms have been identified.

```
relates to the amino acid sequence of isoform 1 of human MEF2C:
SEQ ID NO: 1
                10 20 30 40 50 60
MGRKKIQITR IMDERNRQVT FTKRKFGLMK KAYELSVLCD CEIALIIFNS TNKLFQYAST
                70 80 90 100 110 120
DMDKVLLKYT EYNEPHESRT NSDIVETLRK KGLNGCDSPD PDADDSVGHS PESEDKYRKI
              130 140 150 160 170 180
NEDIDLMISR QRLCAVPPPN FEMPVSIPVS SHNSLVYSNP VSSLGNPNLL PLAHPSLQRN
              190 200 210 220 230 240
SMSPGVTHRP PSAGNTGGLM GGDLTSGAGT SAGNGYGNPR NSPGLLVSPG NLNKNMQAKS
              250 260 270 280 290 300
PPPMNLGMNN RKPDLRVLIP PGSKNTMPSV SEDVDLLLNQ RINNSQSAQS LATPVVSVAT
              310 320 330 340 350 360
PTLPGQGMGG YPSAISTTYG TEYSLSSADL SSLSGFNTAS ALHLGSVTGW QQQHLHNMPP
              370 380 390 400 410 420
SALSQLGACT STHLSQSSNL SLPSTQSLNI KSEPVSPPRD RTTTPSRYPQ HTRHEAGRSP
              430 440 450 460 470
VDSLSSCSSS YDGSDREDHR NEFHSPIGLT RPSPDERESP SVKRMRLSEG WAT
```

Out of the 473 amino acids, approximately 20-50 amino acids are unique to MEF2C. From this unique stretch of amino acids a 13 amino acid sequence from human MEF2C was selected for use in generating the monoclonal antibodies of the present invention. This particular peptide sequence (epitope) was chosen due to its lack of secondary structure and absence of internal cysteines and its low sequence similarity with other MEF2 family proteins, thus ensuring the selectivity of the monoclonal antibodies of the present invention for the MEF2C protein only. Beneficially, this 13 amino acid sequence is highly conserved across mammals, including rodents and non-rodents, meaning that the monoclonal antibodies of the present invention would be expected to bind to other mammalian MEF2Cs in addition to human MEF2C.

The 13 amino acid sequence from human MEF2C (SEQ ID NO: 2) is as follows:

GNPNLLPLAHPSL

Differentiation of Stem Cells into Other MEF2C Expressing Cell Types

MEF2C is also a known marker of differentiation of stem cells that are destined for lineages other than cardiopoiesis. Cells and tissues in vascular development are known to express MEF2C, and the factor is known to play an important role in neural stem cell development, neurogenesis and especially in brain development (Li et al. (2008) Proc Natl Acad Sci USA. July 8; 105(27): 9397-9402). By way of example, haploinsufficiency of MEF2C in humans is believed to be responsible for severe mental retardation with stereotypic movements, seizures and/or cerebral malformations (Le Meur et al. (2010) J. Med. Genet. January 47(1):22-29). Misregulation of MEF2C is also implicated in developmental pathways that can lead to complex craniofacial defects and hearing loss characterised by so-called Waardenburg syndromes (Agarwal et al. (2011) Development, 138(12):2555-2565). Hence, methods, kits and compositions that allow better monitoring of MEF2C expression and localisation can play a significant role in diagnostics, research and biomonitoring outside of and in addition to the context of cardiopoiesis.

Generation of Antibodies

Disclosed herein are isolated antibodies or fragments thereof, in particular isolated monoclonal antibodies or fragments thereof, that are able to bind to MEF2C and in particular to a specific epitope of human MEF2C.

Monoclonal antibodies of the invention can be produced using conventional monoclonal antibody generation techniques.

One way to generate the monoclonal antibodies of the invention is to immunise a mouse with the human MEF2C antigen comprising the amino acid sequence of SEQ ID NO: 2. After the mouse has mounted an immune response to the antigen, i.e. by producing lymphocytes expressing antibodies against the antigen, spleen cells are removed from the immunised mouse and are fused to a specialised myeloma cell line that no longer produces an antibody of its own. The resulting fused cells are known as hybridomas and are able to produce the desired antibody whilst also being able to grow indefinitely in a suitable selective medium—thus the desired antibody is available in limitless quantities.

Other known methods for generating monoclonal antibodies may be used, such as by using rabbit B-cells to form a rabbit hybridoma.

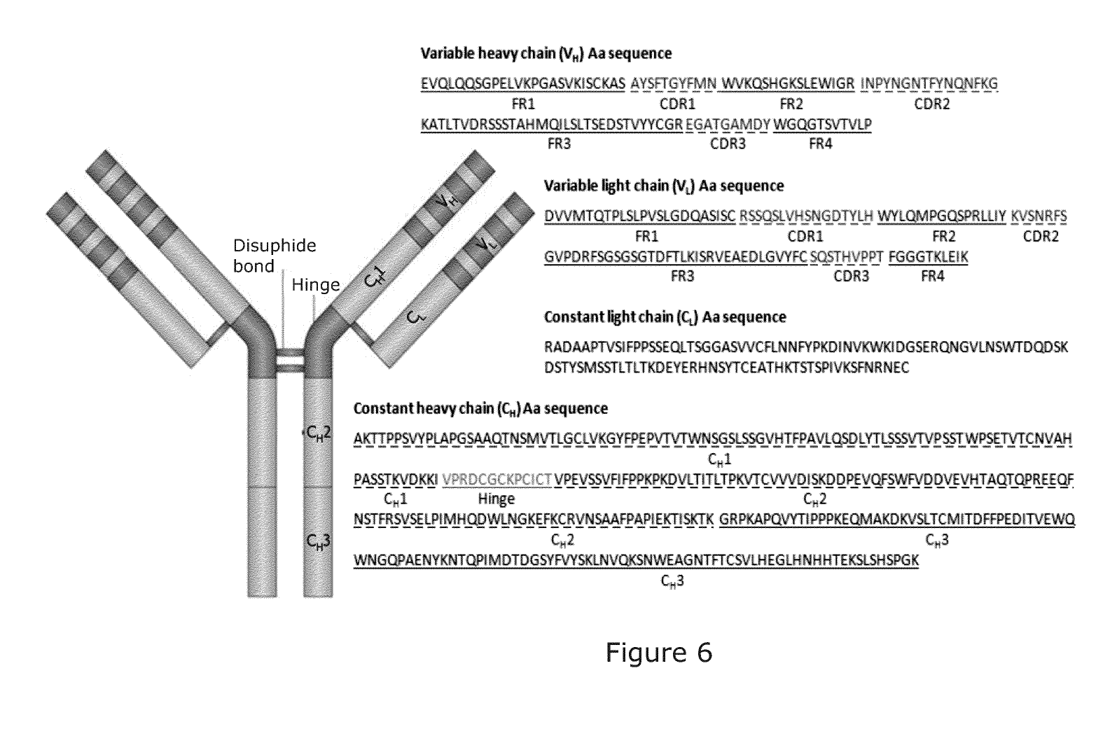

The monoclonal antibodies of the present invention may be produced by a hybridoma cell line such as that deposited by the present applicant with BCCM/LMBP on 20 Dec. 2012 and having contract number BCCM/LMBP/12-16, corresponding to Accession No. LMBP 9949CB (BCCM/LMBP, Plasmidcollectie Vakgroep Moleculaire Biologie, Universiteit Gent, Technologiepark 927, B-9052 Gent-Zwijnaarde, Belgium). The monoclonal anti-MEF2C antibodies of the invention produced by the aforementioned deposited hybridoma produce IgG antibodies having nucleic and amino acid sequences of heavy and light chains as set out in FIG. 6 and as listed in SEQ ID NOs: 5-12. Accordingly, the present disclosure provides, for example, antibodies or antigen binding fragments thereof, comprising a heavy chain variable region polypeptide having at least 80%, 85%, 90%, 95%, or greater amino acid sequence identity to an amino acid sequence of a heavy chain variable region described herein (e.g., SEQ ID NO: 6), and a variable light chain polypeptide having at least 80%, 85%, 90%, 95%, or greater amino acid sequence identity to an amino acid sequence of a light chain polypeptide as set forth herein (e.g., SEQ ID NO: 8). In an embodiment of the invention the monoclonal antibodies may comprise only the heavy chain complementarity-determining region (CDR) selected from at least one or more of SEQ ID NOs: 13-15, and the light chain comprises a CDR selected from at least one or more of SEQ ID NOs: 16-18.

The monoclonal antibodies of the present invention are able to bind to human MEF2C with unexpected and exceptionally high specificity and sensitivity and are thus able to readily detect cardiovascular lineage committed cells. Monoclonal antibodies typically tend to have greater selectivity but lower sensitivity than polyclonal antibodies, however the monoclonal antibodies of the invention buck this trend by having both high selectivity and high specificity. Compared to rabbit polyclonal antibodies, which typically require a dilution of approximately 1:150 of a 0.21 mg/ml to 0.73 mg/ml stock to be able to detect MEF2C, the monoclonal antibodies of the invention can detect MEF2C at a dilution of approximately 1:3000 of a 3 mg/ml stock—thus displaying a sensitivity and potency of approximately 1.5 to 5× that of the rabbit polyclonal antibodies.

Monoclonal antibodies of the present invention are shown to demonstrate an equilibrium dissociation constant (K_{d}) at least in the nanomolar range—i.e. at least 10⁻⁷ M. The K_{d} represents the ratio of the antibody dissociation rate (k_{off}), how quickly it dissociates from its antigen, to the antibody association rate (kₒₙ) of the antibody, how quickly it binds to its antigen.

The antigen used to immunise the mice for monoclonal antibody generation, namely that comprising the amino acid sequence of SEQ ID NO:2, shows very low similarity with other MEF2 family proteins, thus ensuring the high selectivity of the monoclonal antibodies for the MEF2C protein. The amino acid sequence of SEQ ID NO: 2 is also highly conserved across mammals, including rodents and non-rodents. Thus, although the monoclonal antibodies of the present invention have been primarily tested for their ability to bind to human MEF2C, it is expected that they would also be capable of binding to MEF2C in other mammals with similarly high specificity and sensitivity. The antibodies of the present invention show particular advantage in identifying cells derived from adult or embryonic stem cells that show the highest level of commitment to the cardiovascular lineage.

Mapping of the epitope described in SEQ ID NO: 2 showed that six of the seven first amino acids, namely the sequence GNPNLxL, are required for the binding of an anti-MEF2C monoclonal antibody according to one embodiment of the present invention. Alanine mutation of any of those first seven amino acids was seen to result in a relative binding decrease of the anti-MEF2C by more than 90%. Therefore, in one embodiment of the present invention, the epitope targeted by the anti-MEF2c monoclonal antibody is defined as the amino acid sequence:

[SEQ ID NO: 3]
GNPNLxL

where uppercase and lowercase letters indicate according to the one letter amino acid code, respectively, critical and important amino acids for the epitope recognition by the anti-MEF2C monoclonal antibody and x non-specific amino acid residues for that purpose.

A seventh amino acid residue, namely the serine at the twelfth position of the peptide, was also determined as having some importance since its mutation decreased the relative binding of the anti-MEF2C monoclonal antibody by more than 50%. Therefore, in a further embodiment of the present invention, the epitope targeted by the anti-MEF2C monoclonal antibody is defined as the amino acid sequence:

[SEQ ID NO: 4]
GNPNLxPxxxxs

Antibody fragments capable of binding to the epitope determinants described herein, such as an Fv fragment, an Fab fragment, or an F(ab′)₂ fragment, can be prepared according to conventional methods in the art, such as by proteolytic hydrolysis of the antibody or by expression in a host cell of DNA encoding the fragment. Other chemical, enzymatic or genetic techniques may be used to cleave the antibodies to generate fragments, so long as the fragments bind to the antigen that is recognised by the whole antibody.

Conservative variants of the antibodies or antibody fragments of the invention, i.e. those generated by substituting one or more amino acids of the antibody or antibody fragment with a functionally similar amino acid, can be produced using conventional techniques. The conservative variants will typically bind the target antigen with an equal efficiency or possibly even greater efficiency than the parent antibody or parent antibody fragment.

Effector molecules, such as therapeutic, diagnostic, or detection moieties can be linked to the antibodies or antibody fragments of the invention using any method known in the art.

Also disclosed are nucleic acids encoding the amino acid sequences of the antibodies or antibody fragments of the invention. Nucleic acids encoding antibodies produced by the hybridoma cell line of the present invention can readily be produced by one of skill in the art using the amino acid sequences disclosed herein by any suitable method known in the art—for example, by direct chemical synthesis methods, by cloning of appropriate sequences and/or by amplification methods. Furthermore, a variety of clones containing functionally equivalent nucleic acids but which encode the same antibody sequence or antibody fragment sequence can readily be constructed by one of skill in the art.

In addition to conventional methods of antibody generation, the MEF2C epitope (such as that encoded by SEQ ID NOs:2-4) may be used to screen one or more peptide (i.e. antibody) display libraries in order to identify and isolate clones that encode antibodies with a high binding affinity for the peptide. Suitable peptide display library expression systems that may be used include, but are not limited to, in vitro peptide generation libraries, such as: mRNA display (Roberts, & Szostak (1997), Proc. Natl. Acad. Sci. USA, 94, 12297-12302); ribosome display (Mattheakis et al., (1994), Proc. Natl. Acad. Sci. USA, 91, 9022-9026); and CIS display (Odegrip et al., (2004), Proc. Natl. Acad. Sci USA, 101 2806-2810). Alternatively, phage display systems including human combinatorial antibody libraries (e.g. HuCAL®, MorphoSys AG, Germany) may be screened to identify suitable high affinity binding antibodies.

The monoclonal antibodies of the invention may also be subjected to in vitro affinity maturation in order to further optimise binding specificity against the epitopes encoded by SEQ ID Nos: 2-4. In this process the sequence of SDRs in the antibody may be subjected to random or directed mutagenesis in order to generate variants with even higher binding specificity when screened against antigen comprised within a peptide display library (for example, see De Pascalis et al. Clin Cancer Res. (2003) 15; 9(15):5521-31). Monoclonal antibodies obtained by way of in vitro affinity maturation are able to compete for epitope binding with antibodies produced by hybridomas, and as such also fall within the scope of the present invention.

Nucleic acids encoding the antibodies or antibody fragments of the invention can be expressed in recombinant host cells according to conventional techniques (Sambrook J. et al, Molecular Cloning: a Laboratory Manual, Cold Spring Harbor Press, Cold Spring Harbor, N.Y.). Suitable host cells are those that can be grown in culture and are amenable to transformation with exogenous DNA, including bacteria, fungal cells, insect cells and cells of higher eukaryotic origin, preferably mammalian cells.

To enable expression in a host cell, the nucleic acids encoding the antibodies or antibody fragments of the invention can be operatively linked to expression control sequences, including appropriate promoters, enhancers, transcription terminators, start codons and stop codons, etc., and inserted into an expression vector. Suitable expression vectors include plasmids and viruses or any other vehicle that can be manipulated to allow insertion or incorporation of the nucleic cod sequences in a host cell.

Methods of stable transfer enabling the foreign DNA, suitably contained within an expression vector, to be continuously maintained in the host cell, whether it be prokaryotic or eukaryotic, are known in the art.

Once expressed, the antibody polypeptides of the invention or fragments thereof, can be isolated and purified using standard procedures in the art, including for example chromatography, precipitation and immunological separations.

In addition to recombinant methods, the antibodies of the invention and fragments thereof, may also be constructed in whole or in part using standard peptide synthesis techniques.

Detecting Cardiovascular Lineage Committed Cells and Quantification of Cardiovascular Lineage Commitment

Disclosed herein are methods for detecting and/or isolating and/or quantification cardiovascular lineage committed cells.

The methods include contacting a biological sample with the antibodies of the invention, or fragments thereof, under conditions where an immune complex will form between the antibody, or fragment thereof, and the target antigen—MEF2C. The presence (or absence) of the immune complex is then detected and can also be used to isolate cells of interest as well as can be used for quantification The presence of the immune complex indicates the presence of a cardiovascular lineage committed cell, quantification can measure the commitment towards the cardiovascular lineage.

The biological sample can be any sample that potentially includes cardiac lineage committed cells. For example, the biological sample may include a sample of adult stem cells, embryonic stem cells, induced pluripotent stem cells (IPS), marrow-isolated adult multilineage inducible cells (MIAMI), resident cardiac stem cells, or any combination thereof, either or not having been subjected to a cardiogenic cocktail of substances that has caused differentiation of the cells into cardiac lineage committed cells. Suitably, the stem cells may be harvested from a suitable tissue source including, for example, bone marrow, adipose tissue, umbilical cord blood, amniotic fluid, menstrual fluid and blood. Suitably, cells committed to the generation of heart tissue are mammalian cells, typically selected from the group consisting of humans, cats, dogs, pigs, horses, mice, rats, hamsters and other mammals, preferably humans.

The methods are typically performed in vitro on a biological sample obtained from a subject. The cells of the biological sample are typically lysed/disrupted prior to detection and/or quantification with the antibodies of the invention so that the antibodies can access the target MEF2C epitope of SEQ ID NO: 2. The antibodies of the invention, or fragments thereof, are then added to the disrupted biological sample and if the MEF2C antigen is present an immune complex will form between the antibodies and the antigen.

Methods and antibodies according to the present invention are particularly suitable for ensuring quality control for products that comprise cellular preparations that comprise cardiac lineage committed (cardiopoietic cells). Several quality control release tests are routinely performed on manufactured batches of such cellular preparations, including:
Identity
Unintended cell-type impurity levels
Homogeneity

In batches of cell preparations that pass both purity and homogeneity criteria, cardiopoietic cells show a characteristic nuclear translocation of MEF2C which is suitably detected by immunofluorescence with a specific anti-MEF2C monoclonal antibody (mAb) according to the present invention.

The antibodies of the present invention may be labelled with or coupled to a detectable agent to enable detection and/or quantification of the immune complex, as per conventional direct detection and/or quantification methods. However, typically, the biological sample containing the immune complex will be contacted with a second (secondary) antibody that specifically binds the (first/primary) antibody, or fragment thereof, of the present invention, wherein the second antibody is labelled with a detectable and/or quantifiable agent or probe. The second antibody may be a polyclonal or a monoclonal antibody and may suitably be of mammalian, human, goat, rabbit, rodent or chimeric origin, for example. The type of detectable and/or quantifiable agent will depend upon the method of detection or immunoassay used to detect and/or quantify the cardiovascular lineage committed cells. Immunoassays suitable for the detection and/or quantification of differentiated cells include, for example, immunofluorescence, immunohistochemistry, immunoprecipitation, Western blotting and enzyme-linked immunosorbant assays (ELISA). These assays are well known to the person of skill in the art, see for example Sambrook J. et al, Molecular Cloning: a Laboratory Manual, Cold Spring Harbor Press, Cold Spring Harbor, N.Y.

Suitable detectable and/or quantifiable agents or probes coupled to the second antibody, or indeed the first antibody, include, for example: biotin; reporter enzymes such as alkaline phosphatase or horseradish peroxidase; fluorescent labels; luminescent labels; radioactive labels; and chromogenic labels, amongst others.

For example, enzyme immunoassays may be performed using peroxidase, alkaline phosphatase, β-galactosidase, urease, catalase, glucose oxidase, lactate dehydrogenase, amylase, a biotin-avidin complex, or the like as probes/detectable agents. Fluorescent immunoassays may be performed using a fluorescent substance or a fluorophore, such as fluorescein isothiocyanate, tetramethylrhodamine isothiocyanate, substituted rhodamine isothiocyanate, dichlorotriazine isothiocyanate, Alexa, or AlexaFluoro, and fluorescent proteins including GFP and phycoerythrin as probes/detectable agents. Examples of radioisotopes useful for radioimmunoassays include tritium, iodine (¹³¹I, ¹²⁵I, ¹²³I, and ¹²¹I) phosphorous (³²P), sulphur (³⁵S), and metals (e.g., ⁶⁸Ga, ⁶⁷Ga, ³⁸Ge, ⁵⁴Mn, ⁹⁹Mo, ⁹⁹Tc, ¹³³Xe, etc.). Luminescent immunoassays may suitably be carried out with a luciferase system, a luminol-hydrogen peroxide-peroxidase system, a dioxetane compound system, for example.

In the context of cardiopoiesis positive detection and/or quantification of an immune complex between the antibodies of the present invention and the MEF2C antigen indicates the presence of a cardiovascular lineage committed cell. By analogy in other contexts, such as neurogenesis, positive detection and/or quantification of an immune complex between the antibodies of the present invention and the MEF2C antigen indicates the presence of other specified cell types, such as neural cells.

Therefore, a method for detecting and/or quantifying a cardiovascular lineage committed cell, may comprise:

differentiating cells, including stem cells in a biological sample towards a cardiac cell lineage using a composition comprising one or more growth factors, cytokines, hormones and/or combinations thereof;
contacting the biological sample with the antibody, or fragment thereof, of the present invention under conditions where an immune complex will form between the antibody, or fragment thereof, and the target antigen on the differentiated stem cell; and
detecting and/or quantifying the presence of the immune complex;
wherein the presence of the immune complex indicates the presence of a cardiovascular lineage committed cell and/or quantifies its commitment to the cardiovascular lineage.

Likewise, a method for detecting and/or quantifying a neural lineage committed cell, may comprise:
differentiating cells, including stem cells in a biological sample towards a neural cell lineage using a composition comprising one or more growth factors, cytokines, hormones and/or combinations thereof;
contacting the biological sample with the antibody, or fragment thereof, of the present invention under conditions where an immune complex will form between the antibody, or fragment thereof, and the target antigen on the differentiated stem cell; and
detecting and/or quantifying the presence of the immune complex;
wherein the presence of the immune complex indicates the presence of a neural lineage committed cell and/or quantifies its commitment to the neural lineage.

In specific embodiment of the invention the neural cell lineage is a neuronal cell lineage, optionally a doperminergic neuron cell.

Suitably, the target antigen is the human MEF2C epitope of any one of SEQ ID NOs: 2-4.

Compositions and Therapeutic Methods

Disclosed herein are compositions comprising the antibodies of the invention, or fragments thereof, for use in the detection and/or quantification of cardiovascular lineage committed cells, neural lineage committed cells, and also neuronal cells. The compositions can be formulated with an appropriate liquid or solid carrier depending upon the particular mode of administration chosen. Typically, the compositions will be prepared with a liquid carrier and used in vitro to detect and/or quantify suitably differentiated cells, including stem cells, namely cardiovascular lineage committed cells, expressing the MEF2C biomarker. One such suitable composition may comprise antibodies of the invention and a PBS/glycerol carrier.

Also disclosed herein is a method of treating an individual having a heart disorder or disease. As used herein, “treat” or “treatment” means alleviation of or a postponement of development of the symptoms associated with a disease or disorder described herein. The terms further include ameliorating existing uncontrolled or unwanted symptoms, and preventing additional symptoms, Hence, the terms denote that a beneficial result has been conferred on an individual suffering from a disease or symptom, or with the potential to develop such disease or symptom. A response is achieved when the individual experiences at least some alleviation, or reduction of signs or symptoms of illness, and specifically includes, without limitation, prolongation of predicted survival. The expected progression-free survival times can be measured in months to years, depending on prognostic factors including the number of relapses, stage of disease, and other factors.

Cell-based reparative approaches are increasingly being considered in the management of ischemic heart disease. In particular, therapies are being developed to guide naïve human stem cells towards a cardiac lineage prior to injection into a patient to aid heart regeneration. It has been demonstrated that cardiogenic cocktail-induced cell differentiation of bone marrow derived human mesenchymal stem cells ensures safe and lasting functional and structural benefit following transplant into a failing murine ischemic heart (Behfar et al., Guided Cardiopoiesis Enhances Therapeutic Benefit of Bone Marrow Human Mesenchymal Stem Cells in Chronic Myocardial Infarction, Journal of the American College of Cardiology, Vol. 56, No. 9, 2010).

The method of treating an individual having a heart disorder comprises obtaining cells, including stem cells, from an individual; differentiating the stem cells towards a cardiovascular lineage; detecting and/or quantifying whether the stem cells have become cardiovascular lineage committed cells using the isolated antibodies of the present invention, or fragments thereof; isolating the cardiovascular lineage committed cells; and administering the cardiovascular lineage committed cells to an individual in need thereof.

Further disclosed is a method of treating an individual having a neurodegenerative disorder comprising obtaining cells, including stem cells, from an individual; differentiating the stem cells towards a neural or neuronal lineage; detecting and/or quantifying whether the stem cells have become neural or neuronal lineage committed cells using the isolated antibodies of the present invention, or fragments thereof; isolating the committed cells; and administering the neural or neuronal lineage committed cells to an individual in need thereof. Neurodegenerative diseases may include, but are not limited to, diseases that lead to the progressive loss of or malfunction of neurons. Exemplary neurodegenerative diseases include Parkinson's disease, Alzheimer's disease, Huntington's disease and Amyotrophic lateral sclerosis (ALS).

Kits

Also disclosed herein are kits containing the antibodies of the invention, or fragments thereof, for use in the detection and/or quantification of cardiovascular lineage, neural or neuronal committed cells, typically in vitro. Hence, the antibodies and kits of the invention are suitably used in methods to measure the level of commitment of cells to a cardiopoietic or other lineage in which MEF2C expression or localisation is an indicative factor.

The kits may include instructional materials disclosing the means of use of the antibodies of the invention or fragments thereof in detecting and/or quantifying suitably differentiated stem cells. The instructional materials may be in written or electronic form, for example.

The kits may also include buffers, a carrier and/or reagents suitable for use in the method of detection and/or quantification. Furthermore, the kits may also include a control sample/reaction for verification that the method of detection and/or quantification is working correctly.

In addition, the kits may include additional components required to perform an immunoassay. The additional components will vary depending on the type of immunoassay to be used. For example, the antibodies of the invention or fragments thereof may be directly or indirectly conjugated to other compounds including enzymes, haptens, fluorochromes, metal compounds, radioactive compounds, drugs or magnetic beads. The immunoassays may include radioimmunoassays, enzyme-linked immunosorbant assays (ELISA), immunoprecipitation assays, immunohistochemical assays, Western blotting and immunofluorescence. In the case of Western blotting, for example, the kits may additionally include secondary antibodies coupled to a chemiluminescent agent to detect the binding of the antibodies of the present invention or fragments thereof to the MEF2C biomarker. In the case of immunofluorescence, for example, the kits may additionally include secondary antibodies bound to fluorescent agents to detect and/or quantify the binding of the antibodies of the present invention or fragments thereof to the MEF2C biomarker.

### EXAMPLES

### Example 1

### Detection of MEF2C by Western Blotting

Cell extracts (2 μg/lane) of empty vector (−) or MEF2C coding vector (+) transfected cells were separated on SDS-PAGE (10% acrylamide/bisacrylamide) at 20 mA per gel. Proteins were transferred onto nitrocellulose membrane (Hybond C Extra nitrocellulose, Amersham, #RPN203E) for 90 minutes at 60 V.

After electroblotting, membranes were saturated with blocking buffer [TBS, 0.1% Tween, 5% Blotting Grade Blocker (Bio-Rad, #170-6404)] for 30 minutes at room temperature. This was followed by incubation with the primary antibody of the present invention, i.e. monoclonal antibody from hybridoma cell line BCCM/LMBP/DBT1/12-16, diluted at 1:500 in blocking buffer, overnight at 4° C. under gentle agitation.

After washes in TBS-T 0.1%, membranes were incubated at room temperature under gentle agitation, with appropriate secondary antibody linked to horseradish peroxidase reporter enzyme and diluted (1:2000) in blocking buffer.

After washes in TBS-T, detection was performed by chemoluminescence (Perkin Elmer Plus ECL, # NEL103001 EA) with Hyperfilm ECL (Amersham, #28906837).

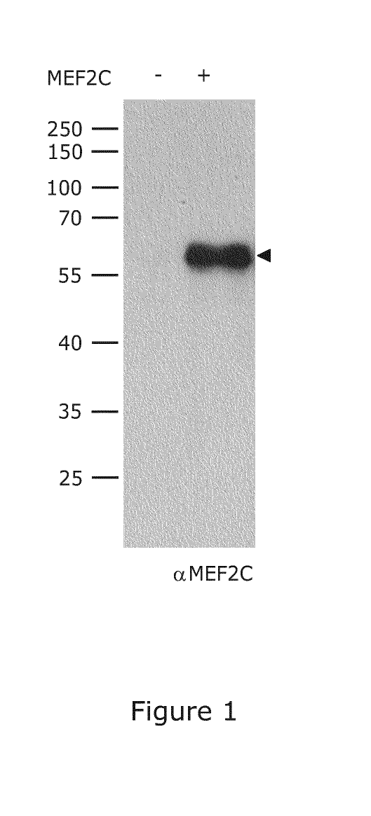

FIG. 1 shows the results of this immunodetection using Western blotting. As indicated by the small arrow, the anti-MEF2C monoclonal antibody (a MEF2C) of the present invention recognises a specific band in the MEF2C transfected cells (+) at around 60 kDa—this corresponds to MEF2C.

### Example 2

### Detection of MEF2C by Immunofluorescence

Specificity of the monoclonal anti-MEF2C antibody of the invention for its target recognition in an immunofluorescence assay was evaluated on HeLa cells transfected with Flag-tagged full length MEF2C protein.

Briefly, HeLa cells were transiently transfected, in 6 well plates, with 1.5 μg of commercial plasmid(Origene MEF2C (NM_{—}002397) Human cDNA ORF Clone with QIAGEN PolyFect transfection reagent). Twenty four hours later, cells were harvested and seeded into biocoated slides at a concentration of 50,000 cells/well.

After overnight culture, cells were fixated in PBS PFA 3% solution then permeabilised in PBS Triton 1% solution.

Cells were blocked with undiluted Superblock for 30 minutes at room temperature, the blocking step was followed by incubation with 1:250 of rabbit anti-Flag (Sigma Monoclonal ANTI-FLAG® M2 antibody produced in mouse F1804—1 mg/ml,) and the primary antibody of the present invention, i.e. monoclonal antibody from hybridoma cell line BCCM/LMBP/DBT1/12-16, 3 mg/ml stock diluted 1:3000, in blocking buffer overnight at 4° C.

After washes in PBS Tween 20 0.1%, cells were incubated with 1:500 dilution of secondary antibodies (AlexaFluor 488 conjugated goat anti-mouse antibody and Alexa Fluor 568 conjugated goat anti-rabbit IgGs) at room temperature for 1 hour in a humidified box.

After washes in PBS Tween 20 0.1%, slides were mounted with DAPI containing mounting medium and stored at 2-8° C. in the dark for minimum 4 hours.

Picture acquisition was performed within 48 hours post-mounting using NIS-Element BR 3.0 software.

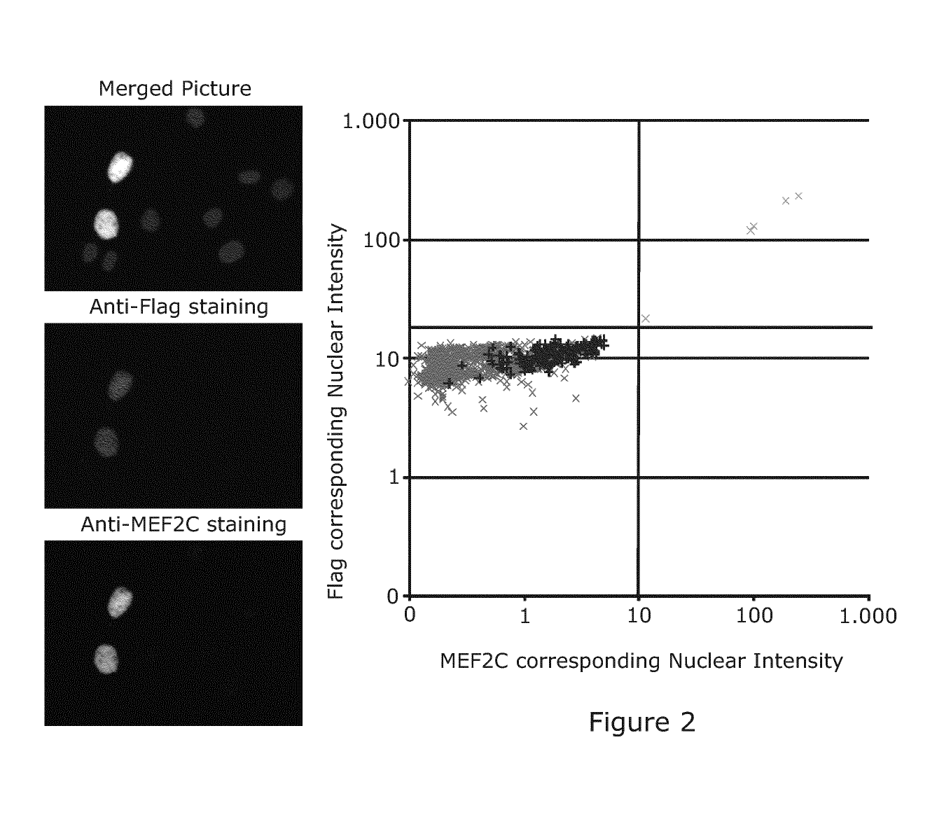

FIG. 2, left panel, shows the results of MEF2C immunostaining in transfected HeLa cells. As shown in the lower picture, the anti-MEF2C monoclonal antibody of the invention was able to detect MEF2C and a signal corresponding to MEF2C was observed in all nuclei positive for the Flag epitope, as shown in the middle picture.

The right panel of FIG. 2 shows the distribution of MEF2C and Flag staining in untransfected (+) and MEF2C transfected (x) HeLa cells. Cut-off values were set based at values of 10 and 20 for MEF2C and Flag staining respectively based on negative cell population staining. Yellow crosses (x) in the top right quadrant of the graph represent double positive nuclei, i.e. cells that show staining intensities for both MEF2C and Flag and exceed the staining of untransfected cells. These cells are considered to be successfully transfected cells since they expressed Flag-tagged MEF2C.

The monoclonal antibodies of the present invention are able to bind to human MEF2C with unexpected and exceptionally high specificity and sensitivity making them excellent candidates for the detection of cardiovascular lineage committed cells. Compared to rabbit polyclonal antibodies, which typically require a dilution of approximately 1:150 of a 0.21 mg/ml to 0.73 mg/ml stock to be able to detect MEF2C, the monoclonal antibodies of the invention can detect MEF2C at a dilution of approximately 1:3000 of a 3 mg/ml stock—thus displaying a sensitivity and potency of approximately 1.5 to 5× that of the rabbit polyclonal antibodies.

### Example 3

### Differentiation of Bone Marrow Mesenchymal Stem Cells into Cardiovascular Lineage Committed Cells

Bone marrow mesenchymal stem cells cultured in Lonza medium were analysed by immunofluorescence using the staining conditions described in Example 2 and compared to cardiovascular lineage committed cells from the same bone marrow origin.

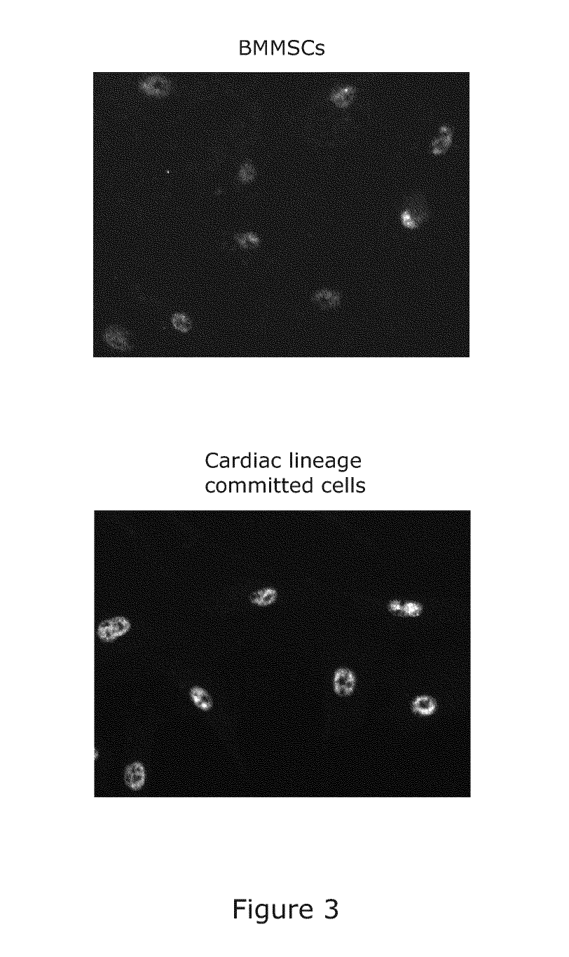

FIG. 3 shows the immunofluorescence of the bone marrow mesenchymal stem cells (left picture) compared to cardiovascular lineage committed cells from the same bone marrow origin (right picture) and clearly shows that most of the nuclei of cardiopoietic cells present an increase in MEF2C biomarker expression compared to naïve mesenchymal stem cells.

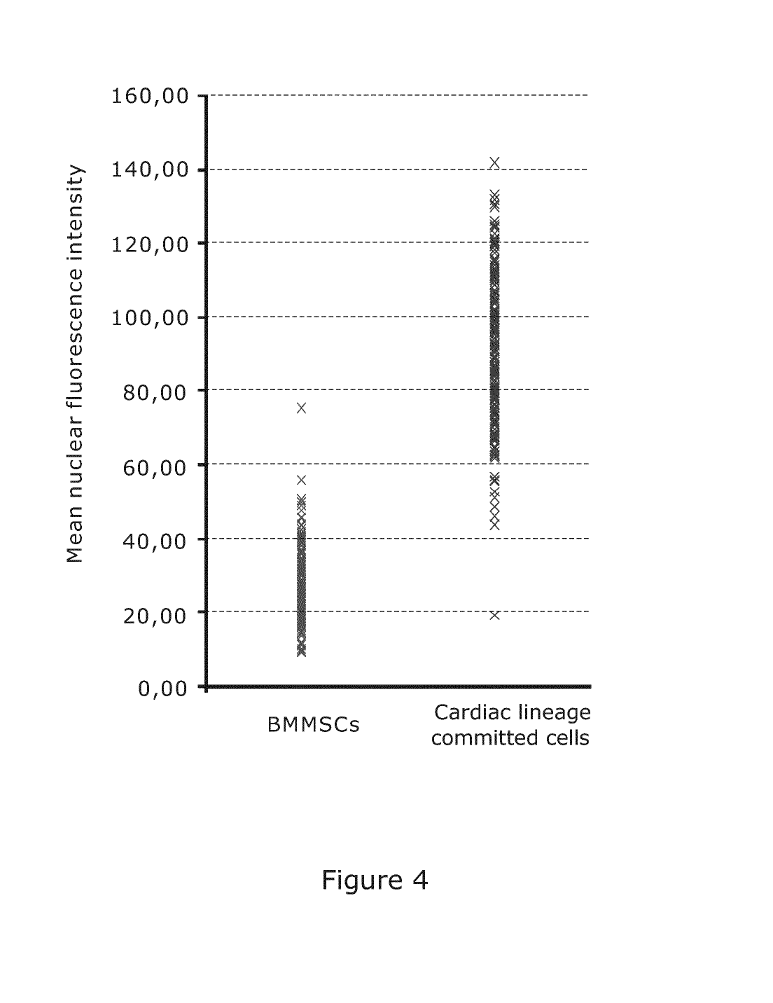

FIG. 4 is a mean nuclear intensity representation of bone marrow mesenchymal stem cell nuclei and cardiovascular lineage committed cell nuclei.

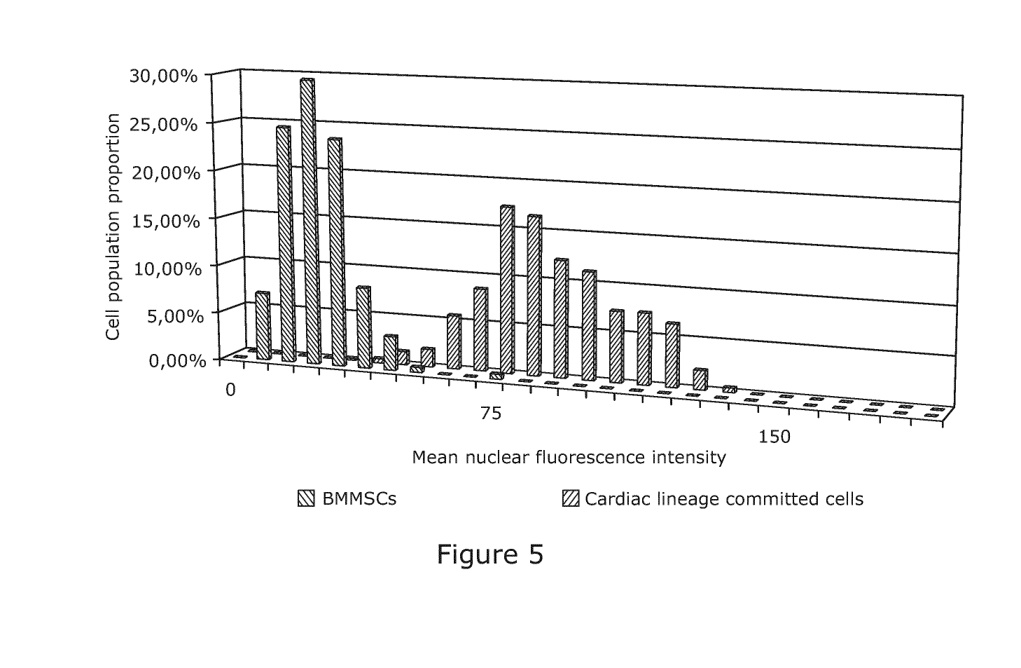

FIG. 5 is a histogram of nuclear fluorescence intensity versus cell population proportion with fluorescence intensity intervals of 7.5 units. Bone marrow mesenchymal cells are represented by the grey front row of bars. Cardiac lineage committed cells are represented by the red back row of bars.

Both FIGS. 4 and 5 demonstrate that most of the nuclei of cardiopoietic cells present an increase in MEF2C biomarker expression compared to naïve mesenchymal stem cells.

This global population shift as regards MEF2C protein expression demonstrates that that the process leading to cardiovascular lineage-commitment effectively leads to an increase of the expression of this widely recognised marker of cardiogenic differentiation.

### Example 4

### Sequencing of Anti-MEF2C Monoclonal Antibody

Total RNA was extracted from frozen hybridoma cells deposited as Accession No. LMBP 9949CB (BCCM/LMBP, Gent, Belgium) and cDNA was synthesized from the RNA. RT-PCR was then performed to amplify the variable regions (heavy and light chains) and constant regions of the antibody, which were then cloned into a standard cloning vector separately and sequenced.

Materials & Methods

Total RNA Extraction:

Total RNA was isolated from the hybridoma cells following the technical manual of TRIzol® (TRIzol® Plus RNA Purification System, Invitrogen, Cat. No.: 15596-026). The total RNA was analyzed by agarose gel electrophoresis.

RT-PCR:

Total RNA was reverse transcribed into cDNA using isotype-specific anti-sense primers or universal primers following the technical manual of SuperScript™ III First-Strand Synthesis System (SuperScript™ III First-Strand Synthesis System, Invitrogen, Cat. No.: 18080-051). The antibody fragments of V_{H}, V_{L}, C_{H} and C_{L} were amplified according to the standard operating procedure of RACE of GenScript.

Cloning of Antibody Genes:

Amplified antibody fragments were separately cloned into a standard cloning vector using standard molecular cloning procedures.

Screening and Sequencing:

Colony PCR screening was performed to identify clones with inserts of correct sizes. No less than five single colonies with inserts of correct sizes were sequenced for each antibody fragment. Five single colonies with correct V_{H}, V_{L}, C_{L} and fourteen colonies with C_{H} insert sizes were sequenced. Unique one kind of V_{H}, V_{L} and C_{L} DNA sequence and three kinds of C_{H} DNA sequences were found. Different clones of each fragment were found to be nearly identical. The consensus sequence shown in FIG. 6 is believed to be the sequence of the anti-MEF2C antibody produced by the hybridoma—denoted as SEQ ID NOs: 5-8.

### Example 5

### Epitope Mapping of the MEF2C Epitope Used to Produce Anti-MEF2C Monoclonal Antibody

This study aims to identify the epitope of anti-MEF2C mAb produced previously. The experiments were designed in line with internationally recognized standards of the technical requirements. The project includes synthesis of an alanine scanning peptide library and detection of the epitope by ELISA.

The peptide of SEQ ID NO: 2 was used as a basis for design of peptide library that would be used to identify the epitope of the target antibody of the invention. The peptide library was synthesized by GenScript®. Peptides were dissolved in DMSO to a final concentration of 10 mg/ml and stored at −20° C.

Additional Reagents and Solutions:
Goat anti-mouse IgG (H+L), 2.3 mg/ml, Thermo, Cat. No.: 31160
Rabbit anti-goat IgG (H+L) [HRP], 0.8 mg/ml, Thermo, Cat. No.: 31402
PBS: NaCl 137 mmol/L,
KCl 2.7 mmol/L,
Na₂HPO₄ 4.3 mmol/L,
KH₂PO₄ 1.4 mmol/L, pH7.4
PBS-T: 0.05% Tween 20 in 1×PBS
Coating buffer: 0.05 M NaHCO₃, pH 9.6
MPBS: 5% skimmed milk in 1×PBS
TMB
1M HCl

ELISA was performed to evaluate binding of the antibody to the antigen. A 96 well microtiter plate was coated with 10 μg/ml of the antigen peptide in 100 μl of coating buffer at 4° C. for 16 h and subsequently blocked with 5% MPBS at 37° C. for 2 h. The plate was washed with PBS-T and incubated with 100 μl target/control antibody at three different concentrations (5 μg/ml, 1 μg/ml, and 0.1 μg/ml) at 37° C. for 2 h. The ELISA plate was then washed with PBS-T and incubated with 100 μl goat anti-mouse IgG (H+L) (0.1 μg/ml) for 1 h. The ELISA plate was then washed with PBS-T and incubated with 100 μl rabbit anti-goat IgG (H+L) [HRP] (0.1 μg/ml) for 1 h. After washing plates with PBS-T, the color was developed with 100 μl TMB substrate for 10 minutes. The reaction was stopped by adding 100 μl of 1 M HCl and the absorbance of each well was measured at 450 nm using a spectrometer.

ELISA was performed to map the epitope of the test antibody. The peptides were coated in 96-well microtiter plates at 4° C. for 16 h. The plates were then blocked with 5% MPBS at 37° C. for 2 h. After washed with PBS-T, 100 μl target/control antibody (0.5 μg/ml) was added and incubated at 37° C. for 2 h. The plates were washed with PBS-T and incubated with 100 μl of 0.1 μg/ml goat anti-mouse IgG (H+L) at 37° C. for 1 h. The plates were washed with PBS-T and incubated with rabbit anti goat IgG (H+L) [HRP] at 37° C. for 1 h. After washing the plate with PBS-T, the color was developed with 100 μl TMB substrate for 10 minutes. The reaction was stopped by adding 100 μl of 1 M HCl and the absorbance was measured at 450 nm using a spectrometer. Positive ELISA signal indicates that the test antibody binds to the peptide. A non-coated well was set as blank control and the peptide library incubated with the detection antibodies only was set as the negative control.

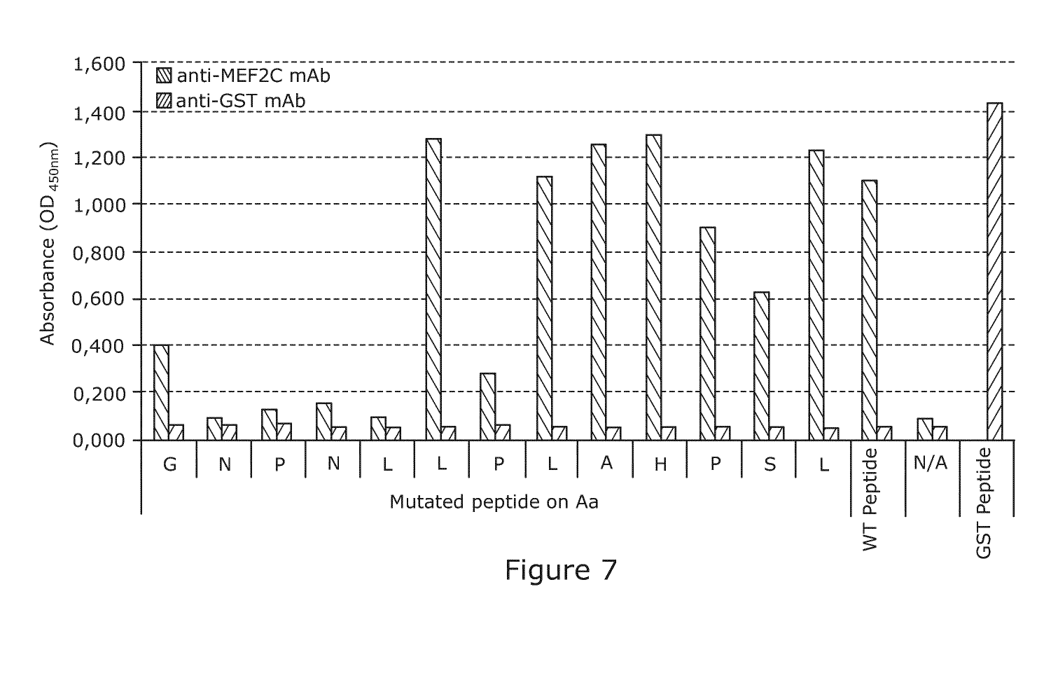

Thirteen mutant peptides were numbered in numerical order and their binding activity with target/control antibodies were determined by indirect ELISA. The non-specific binding between the peptide, the target antibody and rabbit anti-goat IgG [HRP] was determined. The non-specific binding between the peptide, goat anti-mouse IgG and rabbit anti-goat IgG [HRP] was determined as well. As can be seen from the results shown in FIG. 7, mutations at peptide positions 2 through 5 completely abolished the interaction between the antigen peptide and the antibody, indicating that the epitope of the antibody is a stretch of peptide sequence of NPLL. Mutations at peptide positions 1 and 7 also affected the binding, suggesting that the amino acid residues G1″ and P7 may facilitate the binding between the peptide antigen and the antibody.

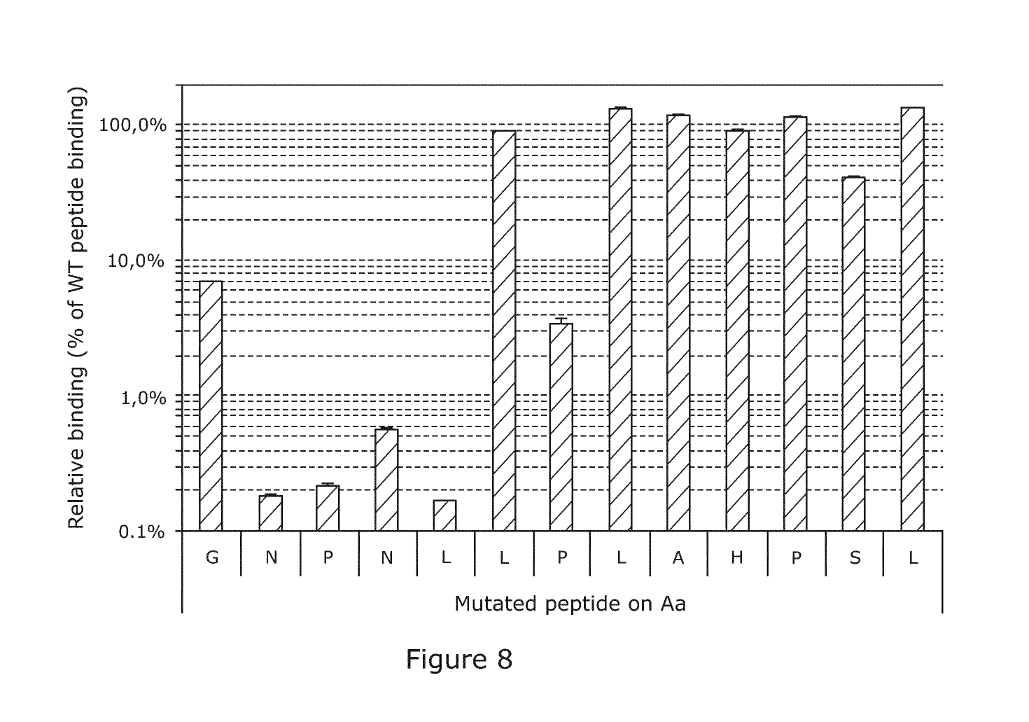

In addition to the alanine scanning, a relative quantification of binding was performed. Each of the mutated peptides was assayed in direct ELISA according to the procedures described above and compared to a standard curve performed with the wild-type peptide. Relative binding quantification was then performed as described previously. The results of the relative quantification of binding are summarized in FIG. 8. As shown in the FIG. 8, mutation of any of the amino acids of the NPNL sequence led to a dramatic decrease of the binding by more than 99%. Moreover, as previously shown, mutations of the first and the seventh residue also impacted the binding with a loss of more than 90% (respectively for the glycine (G) and the proline (P) amino acid replacement by an alanine, a loss of 92.9% and 96.4%). The mutation of the antepenultimate amino acid (S) of the peptide sequence, showed a consistent, although less dramatic, decrease of the binding between the anti-MEF2C monoclonal antibody and the peptide, with a remaining relative binding of 41.3%. Therefore, considering the combination of the data obtained in FIGS. 7 and 8 an epitope target of the anti-MEF2C mAb is the following amino acid sequence:

[SEQ ID NO: 4]
GNPNLxPxxxxs

where uppercase and lowercase letters indicate, respectively, critical and important amino acids for the epitope recognition by the anti-MEF2C mAb and x non-important for that purpose.

### Example 6

### Affinity Measurement of Anti-MEF2C Monoclonal Antibody

Affinity of the antibody for the peptide was measured by using BIAcoreT200. After covalent coupling of the antibody onto the Series S Sensor Chip CM5, different concentrations (0.078, 0.781, 1.563, 3.125 and 6.250 μM) of peptide were injected serially over the antibody and blank flow cell at a flow rate of 30 μl/min. Association and dissociation phases were monitored for 30 and 150 seconds, respectively. Peptide with the concentration of 6.250 μM was set as repetition. The antibody surface was regenerated with 10 mM Glycine-HCl, pH2.0, 30 s injections 1 time.

About 10000 RU of antibody was immobilized on the sensor chip via primary amine groups. Binding data was processed, double referenced with response from blank injections, and fit to a 1:1 interaction model using the BIAcore T200 evaluation software. The kinetics data is shown in Table 1.

TABLE 1

| Ka (1/Ms) | Kd (1/s) | K_{d} (M) |
|---|---|---|
| 2.34 × 10⁴ | 3.67 × 10⁻³ | 1.57 × 10⁻⁷ |

The association constant (Kₐ) could be calculated inverse of K_{d} and was equal to 6.37×10⁶ M⁻¹.

The nanomolar K_{d} value obtained for the monoclonal antibody indicates that it exhibits good specificity for the target epitope.

The results obtained in relation to epitope mapping and affinity measurement taken together with the observed efficacy when used for immunoflorescence detection of MEF2C clearly show that the antibodies of the present invention show considerable advantage in enabling sensitive detection and measurement of MEF2C expression and cellular localisation. The exemplary antibodies of the invention also serve as a base for development of further anti-MEF2C antibodies and related binding peptides using optimised epitopes based on the sequences of SEQ ID Nos: 3 and 4.

Although particular embodiments of the invention have been disclosed herein in detail, this has been done by way of example and for the purposes of illustration only. The aforementioned embodiments are not intended to be limiting with respect to the scope of the appended claims. It is contemplated by the inventor that various substitutions, alterations, and modifications may be made to the invention without departing from the spirit and scope of the invention as defined by the claims.

1. An isolated antibody, or a fragment thereof, that binds selectively to a human MEF2C epitope that consists essentially of an amino acid sequence selected from at least one of: SEQ ID NO: 3 and SEQ ID NO: 4.

2.-41. (canceled)

42. The isolated antibody, or fragment thereof, of claim 1, wherein the epitope comprises the amino acid sequence of SEQ ID NO: 2.

43. The isolated antibody, or fragment thereof, of claim 1, wherein the antibody is a monoclonal antibody.

44. The isolated antibody, or fragment thereof, of claim 1, wherein the antibody is an anti-anti-idiotypic antibody.

45. The isolated antibody, or fragment thereof, of claim 1, wherein the monoclonal antibody is selected from the group consisting of: a mouse monoclonal antibody; a rabbit monoclonal antibody; and a rat monoclonal antibody.

46. The isolated antibody, or fragment thereof, of claim 1, wherein the antibody is a single domain antibody.

47. The isolated antibody, or fragment thereof, of claim 1, where the fragment is an scFv fragment, an scFv₂ fragment, an Fv fragment, an Fab fragment, an Fab′ fragment, or an F(ab′)₂ fragment.

48. A hybridoma cell line as deposited with the Belgian Coordinated Collections of Micro-organisms (BCCM), under accession number LMBP 9949CB that produces the antibody of claim 1.

49. A monoclonal antibody, or fragment thereof, produced by the hybridoma cell line as deposited with the Belgian Coordinated Collections of Micro-organisms (BCCM), under accession number LMBP 9949CB.

50. A monoclonal antibody, or fragment thereof, that is able to compete with a monoclonal antibody, or fragment thereof, produced by the hybridoma cell line as deposited with the BCCM, under accession number LMBP 9949CB, for specific binding to a MEF2C epitope, which epitope consists of an amino acid sequence selected from at least one of: SEQ ID NO: 3 and SEQ ID NO: 4.

51. The monoclonal antibody of claim 50, wherein the heavy chain comprises a complementarity-determining region (CDR) selected from at least one or more of SEQ ID NOs: 13-15, and the light chain comprises a CDR selected from at least one or more of SEQ ID NOs: 16-18.

52. A composition comprising the antibody of claim 1 and a carrier.

53. A method for detecting a cardiovascular lineage committed cell, comprising:
contacting a biological sample that comprises cells with an antibody, or fragment thereof,—of claim 1 under conditions where an immune complex will form between the antibody, or fragment thereof, and a target antigen; and
detecting the presence of the immune complex;
wherein the presence of the immune complex indicates the presence of a cardiovascular lineage committed cell.

54. The method of claim 53, further comprising the step of visualising localisation of the immune complex within the cell.

55. The method of claim 54, wherein the visualisation step is carried out using a technique selected from the group consisting of: light microscopy; UV microscopy; and confocal microscopy.

56. A method for quantifying a cardiovascular lineage commitment of cells, comprising:
contacting a biological sample that comprises cells with an antibody, or fragment thereof of claim 1 under conditions where an immune complex will form between the antibody, or fragment thereof, and a target antigen; and quantifying the presence of the immune complex;
wherein the presence of the immune complex indicates the quantification of cardiovascular lineage commitment of a cell.

57. The method of claim 56, wherein the biological sample is obtained from a human.

58. The method of claim 57, further comprising:
contacting the biological sample with a second antibody that specifically binds the antibody, or fragment thereof.

59. The method of claim 58, wherein the second antibody is coupled to a detectable agent.

60. The method of claim 59, wherein the detectable agent comprises one or more of the group selected from: an enzyme; a fluorescent label; a luminescent label; a radioactive label; or a chromogenic label.

61. A kit for the identification of a cardiovascular lineage committed cell comprising: an antibody, or a fragment thereof, of claim 1, and instructions for using the kit.

62. A method of treating an individual having a heart disorder, comprising;
obtaining cells and/or stem cells from an individual;
differentiating the cells and/or stem cells towards a cardiovascular lineage;
detecting whether the cells and/or stem cells have become cardiovascular lineage committed cells using an antibody, or a fragment thereof, of claim 1;
isolating the cardiovascular lineage committed cells; and
administering the cardiovascular lineage committed cells to an individual in need thereof.


SEQUENCE LISTING (18 sequences)

SEQ ID NO: 1 - protein, 473 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 2 - protein, 13 aa, Homo sapiens
GNPNLLPLAHPSL

SEQ ID NO: 3 - protein, 7 aa, Homo sapiens
  misc_feature (6)..(6): Xaa can be any naturally occurring amino acid
GNPNLXL

SEQ ID NO: 4 - protein, 12 aa, Homo sapiens
  misc_feature (6)..(6): Xaa can be any naturally occurring amino acid
GNPNLXPXXXXS

SEQ ID NO: 5 - DNA, 354 bp, Artificial Sequence
  MONOCLONAL ANTIBODY SEQUENCE
gaggttcagctgcagcagtctggacctgagctggtgaagcctggggcttcagtgaagatatcctgcaaggcttctgcttactcatttactggctactttatgaactgggtgaagcagagccatggaaagagccttgaatggattggacgaattaatccttacaatggtaatactttctacaaccagaatttcaagggcaaagccacattgactgtagacagatcctctagtacggcccacatgcagatcctgagcctgacatctgaggactctacagtctattattgtggaagagagggggctacgggggctatggactactggggtcaaggaacctcagtcaccgtcttgcct

SEQ ID NO: 6 - protein, 118 aa, Artificial Sequence
  MONOCLONAL ANTIBODY SEQUENCE
EVQLQQSGPELVKPGASVKISCKASAYSFTGYFMNWVKQSHGKSLEWIGRINPYNGNTFYNQNFKGKATLTVDRSSSTAHMQILSLTSEDSTVYYCGREGATGAMDYWGQGTSVTVLP

SEQ ID NO: 7 - DNA, 336 bp, Artificial Sequence
  MONOCLONAL ANTIBODY SEQUENCE
gatgttgtgatgacccaaactccactctccctgcctgtcagtcttggagatcaagcctccatctcttgcagatctagtcagagtcttgtacacagtaatggagacacctatttacattggtacctgcagatgccaggccagtctccaaggctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctccgacgttcggtggaggcaccaagctggaaatcaaa

SEQ ID NO: 8 - protein, 112 aa, Artificial Sequence
  MONOCLONAL ANTIBODY SEQUENCE
DVVMTQTPLSLPVSLGDQASISCRSSQSLVHSNGDTYLHWYLQMPGQSPRLLIYKVSNRFSGVPDRFSGSGSGTDFTLKISRVEAEDLGVYFCSQSTHVPPTFGGGTKLEIK

SEQ ID NO: 9 - DNA, 324 bp, Artificial Sequence
  MONOCLONAL ANTIBODY SEQUENCE
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 10 - protein, 107 aa, Artificial Sequence
  MONOCLONAL ANTIBODY SEQUENCE
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - DNA, 975 bp, Artificial Sequence
  MONOCLONAL ANTIBODY SEQUENCE
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 12 - protein, 324 aa, Artificial Sequence
  MONOCLONAL ANTIBODY SEQUENCE
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - protein, 10 aa, Artificial Sequence
  MONOCLONAL ANTIBODY SEQUENCE
AYSFTGYFMN

SEQ ID NO: 14 - protein, 16 aa, Artificial Sequence
  MONOCLONAL ANTIBODY SEQUENCE
INPYNGNTFYNQNFKG

SEQ ID NO: 15 - protein, 9 aa, Artificial Sequence
  MONOCLONAL ANTIBODY
EGATGAMDY

SEQ ID NO: 16 - protein, 16 aa, Artificial Sequence
  MONOCLONAL ANTIBODY SEQUENCE
RSSQSLVHSNGDTYLH

SEQ ID NO: 17 - protein, 7 aa, Artificial Sequence
  MONOCLONAL ANTIBODY SEQUENCE
KVSNRFS

SEQ ID NO: 18 - protein, 9 aa, Artificial Sequence
  MONOCLONAL ANTIBODY SEQUENCE
SQSTHVPPT